(19) United States
(12) Patent Application Publication
(10) Pub. No.: US 2006/0047256 A1
(43) Pub. Date: Mar. 2, 2006

(54) Ostomy belt

(76) Inventor: Florian Levesque, Granby (CA)

Correspondence Address: Louis Tessier, P.O. Box 54029, Mount-Royal, QC, H3P 3H4, CA

(21) Appl. No.: 11/216,135

(22) Filed: Sep. 1, 2005

(30) Foreign Application Priority Data
Sep. 1, 2004 (CA) 2,476,304

(51) Int. Cl. A61F 5/44

(52) U.S. Cl. 604/345

(57) ABSTRACT

A belt for supporting an ostomy pouch. The belt is wearable by an intended user having a waist. The ostomy pouch is attachable to a stoma extending from the intended user. The belt includes a stretchable band for supporting the ostomy pouch, the stretchable band being positionable substantially in register with the waist of the intended user and substantially in register with the ostomy pouch. The stretchable band defining a band lower edge and a susbtantially opposed band upper edge. The stretchable band further defines a pressure exerting direction extending substantially perpendicularly from the band towards the intended user. A first band reinforcement is operatively coupled to at least one of the band lower or upper edges for opposing, at least in part, a substantially circumferential elongation of the stretchable band. The stretchable band is configured, sized and provided with a band elasticity such that: the stretchable band exerts substantially no force onto the ostomy pouch in the pressure exerting direction when the ostomy pouch is substantially empty, and the stretchable band exerts a band force onto the ostomy pouch in the pressure exerting direction when the ostomy pouch is substantially filled, the band force causing an alert pressure to be exerted onto the intended user by the ostomy pouch, the alert pressure being of a magnitude sufficient for alerting the intended user that the pouch is substantially filled.

This Application claims priority from Canadian Patent Application Serial Number 2,476,304 filed Sep. 1, 2004.

### FIELD OF THE INVENTION

The present invention relates generally to the field of ostomy appliance management. More specifically, the present invention is concerned with an ostomy belt.

### BACKGROUND OF THE INVENTION

Some surgical interventions require that at least part of the gastrointestinal tracts or the urinary tracts of a patient be removed. In some of these interventions, the remaining gastrointestinal or urinary tracts are secured to a stoma through which bodily wastes are evacuated. To collect these wastes, a pouch is typically secured to the stoma. A person having undergone such a surgical intervention is referred to as an ostomate.

The pouch typically needs to be continuously attached to the stoma. This results in restrictions to the ostomate's life because of risks of spillage or bursting of the pouch.

In addition, such pouches are typically aesthetically unattractive and tend to reduce the ostomate's self-confidence and self-esteem. Furthermore, intimacy, and more specifically sexual intimacy, is affected by the presence of the pouch.

Attempts at solving the above-mentioned problems have been made and specially adapted belts or ostomy undergarments have been created. However, most prior art ostomy belts and undergarments have many drawbacks. For example, some ostomy belts support the pouch in a substantially horizontal orientation, which results in non-optimal filling of the pouch.

In addition, it is often the case in prior art ostomy belts and undergarments that the pouch is held too firmly against the user, which results in discomfort and risks of bursting of the pouch. Furthermore, some prior art ostomy belts and undergarments position the pouch such that an undesirable pressure is exerted by the pouch on the stoma.

Some prior art belts require that they be closed with the help of fasteners. Therefore, ostomates who have reduced mobility or who are handicapped may find it difficult to properly position and adjust these undergarments.

Another problem encountered by ostomates is that the pouch needs to be worn even while in bed. If the user is in a deep sleep there is a risk that the pouch will fill without waking up the user, which augments risks of spillage and bursting of the pouch.

Against this background, there exists a need in the industry to provide a novel ostomy belt.

An object of the present invention is therefore to provide an improved ostomy belt.

### SUMMARY OF THE INVENTION

In a broad aspect, the invention provides a belt for supporting an ostomy pouch. The belt is wearable by an intended user having a waist. The ostomy pouch is attachable to a stoma extending from the intended user. The belt includes a stretchable band for supporting the ostomy pouch, the stretchable band being positionable substantially in register with the waist of the intended user and substantially in register with the ostomy pouch. The stretchable band defining a band lower edge and a susbtantially opposed band upper edge. The stretchable band further defines a pressure exerting direction extending substantially perpendicularly from the band towards the intended user. A first band reinforcement is operatively coupled to at least one of the band lower or upper edges for opposing, at least in part, a substantially circumferential elongation of the stretchable band. The stretchable band is configured, sized and provided with a band elasticity such that: the stretchable band exerts substantially no force onto the ostomy pouch in the pressure exerting direction when the ostomy pouch is substantially empty, and the stretchable band exerts a band force onto the ostomy pouch in the pressure exerting direction when the ostomy pouch is substantially filled, the band force causing an alert pressure to be exerted onto the intended user by the ostomy pouch, the alert pressure being of a magnitude sufficient for alerting the intended user that the pouch is substantially filled.

Advantageously, the belt is relatively comfortable to wear when the ostomy pouch is not filled. When the ostomy pouch is filled, the alert pressure indicates to the intended user that the ostomy pouch needs to be emptied. In some embodiments of the invention, the added pressure is sufficient for waking up a sleeping intended user, which therefore reduces the risk that the ostomy pouch will burst or spill while the intended user is asleep.

In some embodiments of the invention, the belt hides the pouch and therefore improves intimacy between the intender user and other persons.

In some embodiments of the invention, the stretchable band forms a stretchable band closed loop that is circumferentially uninterrupted. In these embodiments, the belt is therefore relatively easy to position around the waist of the intended user even for intended users suffering from restricted mobility or for handicapped intended users such as, for example, intended users who only have one arm.

In addition, in some embodiments of the invention, the belt extends transversally over a distance of at least twenty centimeters. Such relatively large transversal extensions results in a belt that is relatively easy to maintain at a given position relative to the intended user, which therefore restricts the need to have relatively tight-fitting belts that reduce the comfort of the intended user.

In some embodiments of the invention, the belt includes a pocket for receiving therein the ostomy pouch in a substantially vertical orientation. The pocket is relatively easy to operate by the user and therefore allows the user to relatively easily insert the pouch into the pocket.

Since the pocket maintains the ostomy pouch in a substantially vertical orientation, filling of the pouch is improved relative to some prior art belts wherein the pouch is held substantially horizontally.

In addition, the pocket further helps in immobilizing the ostomy pouch relative to the intended user, which further improves comfort. Also, since the pocket maintains the pouch in a substantially vertical direction, the intended user may relatively easily move, even when lying in bed, with reduced risks of spillage or bursting of the pouch.

Furthermore, the pocket provides a layer of fabric extending between the ostomy pouch and the intended user, which reduces risks of infections caused by a relatively warm and humid environment created on the surface of the skin of ostomy pouch users by a direct contact between the skin and the ostomy pouch.

The belt is relatively inexpensive to manufacture, relatively easy to wash, and relatively easy to use. Also, the belt is relatively compact when folded and therefore relatively easy to carry when not worn by the intended user.

In another broad aspect, the invention provides a belt for supporting an ostomy pouch. The belt is wearable by an intended user having a waist. The ostomy pouch is attachable to a stoma extending from the intended user. The belt includes a stretchable panel for supporting the ostomy pouch, the stretchable panel being positionable substantially in register with the waist of the intended user and substantially in register with the ostomy pouch. The stretchable panel defining a panel lower edge and a susbtantially opposed panel upper edge. The stretchable panel further defines a pressure exerting direction extending substantially perpendicularly from the panel towards the intended user. A first panel reinforcement is operatively coupled to at least one of the panel lower or upper edges for opposing, at least in part, a substantially circumferential elongation of the stretchable panel. The stretchable panel is configured, sized and provided with a panel elasticity such that: the stretchable panel exerts substantially no force onto the ostomy pouch in the pressure exerting direction when the ostomy pouch is substantially empty, and the stretchable panel exerts a panel force onto the ostomy pouch in the pressure exerting direction when the ostomy pouch is substantially filled, the panel force causing an alert pressure to be exerted onto the intended user by the ostomy pouch, the alert pressure being of a magnitude sufficient for alerting the intended user that the pouch is substantially filled.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1, in a perspective view, illustrates a belt in accordance with an embodiment of the present invention;

FIG. 2, in a perspective view, illustrates the belt of FIG. 1 worn by an intended user;

FIG. 3, in a perspective view, illustrates a pocket of the belt of FIG. 1; and

FIG. 4, in a perspective view, illustrates the belt of FIG. 1 worn by an intended user with an ostomy pouch positioned inside the pocket of FIG. 3, the ostomy pouch being substantially filled, which results in an alert pressure being exerted onto the intended user.

### DETAILED DESCRIPTION

FIG. 2 illustrates a belt 10 for supporting an ostomy pouch 12. The belt 10 is wearable by an intended user 13 (partially shown in FIG. 2) having a waist 14. The ostomy pouch 12 is attachable to a stoma 16 extending from the intended user.

This document refers to up and down directions. The reader skilled in the art will readily appreciate that these directions are used only for convenience reasons and refer to directions relative to an intended user 13 who is standing up. However, the use of directional terms should not be interpreted to limit the scopes of the present inventions and it should be understood that it is within the scope of the appended claims to have a belt 10 that is wearable by users who are seated or in a recumbent position, among other positions.

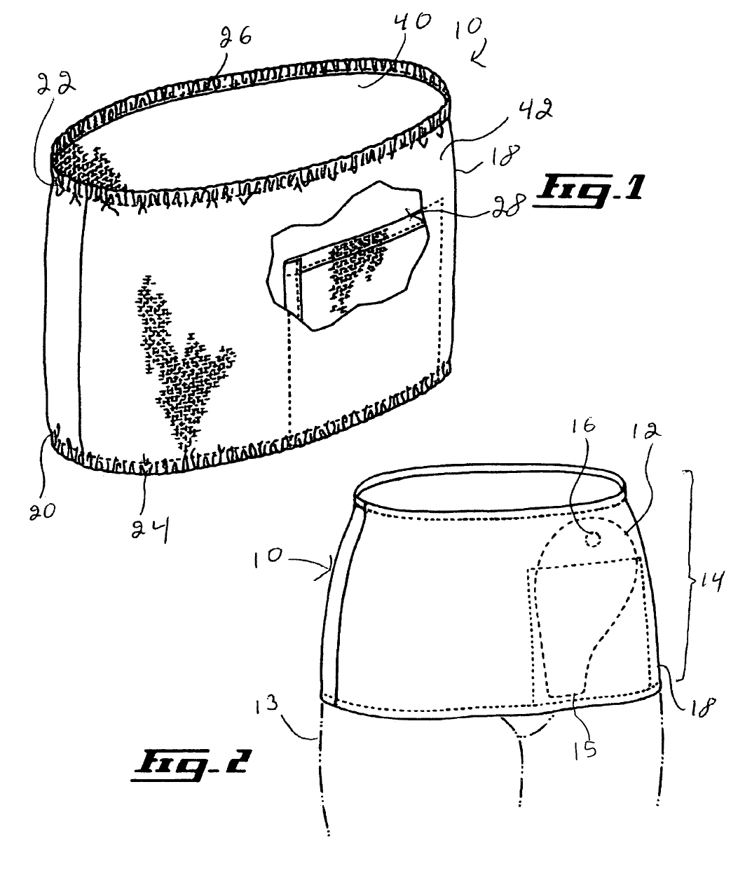

Referring to FIG. 1, the belt 10 includes a stretchable band 18 for supporting the ostomy pouch 12. As seen from FIG. 2, the stretchable band 18 is positionable substantially in register with the waist 14 of the intended user and substantially in register with the ostomy pouch 12. Returning to FIG. 1, the stretchable band 18 defines a band lower edge 20 and a transversally opposed band upper edge 22. The stretchable band 18 further defines a pressure exerting direction extending substantially perpendicularly from the stretchable band 18 towards the intended user 13. Also, the stretchable band 18 defines a band inner surface 40 and an opposed band outer surface 42.

A band reinforcement is operatively coupled to at least one of the band lower or upper edges 24 or 26 for opposing, at least in part, a substantially circumferential elongation of the stretchable band 18. In some embodiments of the invention, the belt 10 includes a first band reinforcement 24 operatively coupled to the band lower edge 20 for opposing at least in part a substantially circumferential elongation of the stretchable band 18. The belt 10 also includes a second band reinforcement 26 operatively coupled to the band upper edge 22 for opposing at least in part a substantially circumferential elongation of the stretchable band 18.

The stretchable band 18 is configured, sized and provided with a band elasticity such that: the stretchable band 18 exerts substantially no force onto the ostomy pouch 12 in the pressure exerting direction when the ostomy pouch 12 is substantially empty, and the stretchable band 18 exerts a band force onto the ostomy pouch 12 in the pressure exerting direction when the ostomy pouch 12 is substantially filled. As seen from FIG. 4, this result in an alert pressure 21 to be exerted onto the intended user 13 by the ostomy pouch 12 when the ostomy pouch 12 is substantially filled, the alert pressure 21 being of a magnitude sufficient for alerting the intended user 13 that the ostomy pouch 12 is substantially filled.

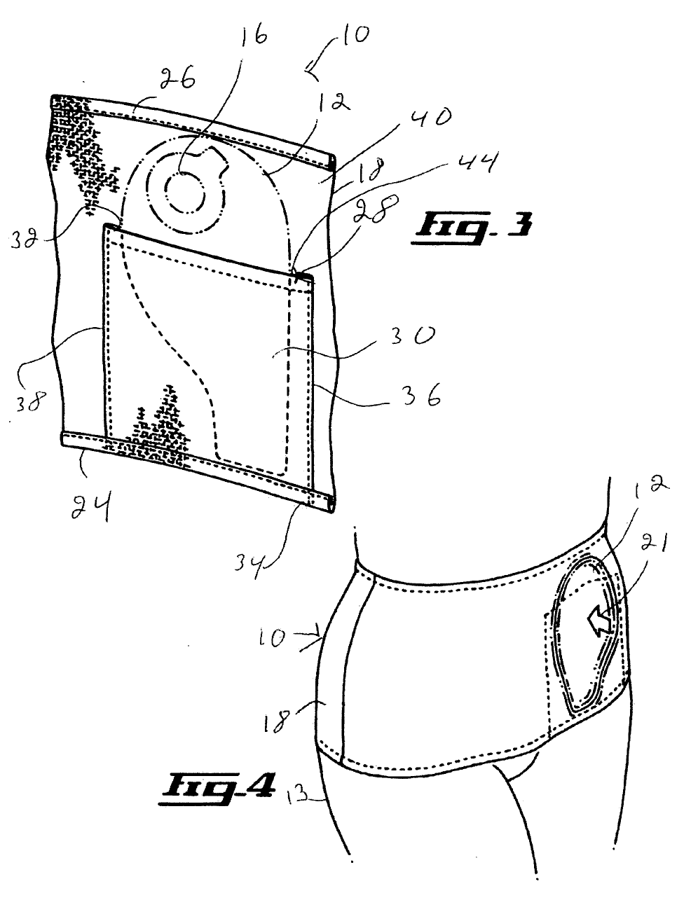

As better seen in FIG. 3, in some embodiments of the invention, a pocket 28 is positionable between the stretchable band 18 and the intended user 13 (not shown in FIG. 3), the pocket 28 being provided for receiving therein the ostomy pouch 12 substantially vertically. However, in other embodiments of the invention, the belt 10 does not include the pocket 28 or includes any other alternative pocket. The pocket 28 is described in further details herein below.

The band elasticity includes a transversal band elasticity and a circumferential band elasticity. In some embodiments of the invention, the transversal band elasticity and circumferential band elasticity are substantially equal. However, in other embodiments of the invention, the transversal band elasticity is substantially smaller than the circumferential band elasticity. In other words, in these embodiments of the invention, a force required to stretch the stretchable band 18 circumferentially by a predetermined percentage is substantially smaller than a force required to stretch the stretchable band 18 transversally by the predetermined percentage.

In some embodiments of the invention, the stretchable band 18 includes a material that is stretchable transversally up to a maximal transversal elongation and is stretchable circumferentially up to a maximal circumferential elongation. The maximal circumferential elongation is substantially larger than the maximal transversal elongation. In some embodiments of the invention, this latter property is linked to having a transversal elasticity and a circumferential band elasticity that are not identical, as described hereinabove.

In some embodiments of the invention, the maximal circumferential elongation is from about 1.1 to about two times larger than the maximal transversal elongation. These individual elongations, in some embodiments of the invention, may take non-limitatively the following values. The maximal circumferential elongation may be from about 100% to 200% while the maximal transversal elongation may be from about 50% to about 150%. Typically, the maximal circumferential elongation is about 150% while the maximal transversal elongation is about 110%.

A material that is suitable for manufacturing the stretchable band 18 is Lycra™. However, the person skilled in the art will readily appreciate that other materials, such as for example nets, may be used to manufacture the belt 10 without departing from the scope of the invention.

Non-limitatively, a Lycra™ that has been found to be suitable for use with the present invention is a Lycra™ having a weight of from about 175 g/m² to about 275 g/m², such as for example a Lycra™ having a weight of about 200 g/m². The use of this type of Lycra™ results in a belt 10 that is relatively porous and therefore allows the belt 10 to breathe so as to eliminate at least in part any moisture that may be produces by the intended user 13 under the belt 10. However, in alternative embodiments of the invention, any other suitable Lycra™ may be used.

It also has been found that having a stretchable band 18 that extends transversally over a distance of at least 20 cm results in a belt 10 that is relatively stable with respect to the intended user 13. Typically, the stretchable band 18 extends in a transversal direction over a distance of from about 20 cm to about 30 cm, and in some specific embodiments of the invention, of from about 22 cm to about 28 cm.

In some embodiments of the invention, the first and second band reinforcements 24 and 26 are resilient and have a reinforcement elasticity. The reinforcement elasticity of the first and second band reinforcements 24 and 26 is substantially smaller than the band elasticity. An example of such first and second band reinforcements 24 and 26 includes elastic ribbons, such as for example and non-limitatively elastic ribbons having a width of about 10 mm.

In some embodiments of the invention a stretchable band closed loop formed by the stretchable band 18 defines a maximal belt circumference. The first and second band reinforcements 24 and 26 each form a respective band reinforcement closed loop secured to the band lower and upper edges 20 and 22. The band reinforcement closed loops have a band reinforcement loop length that is substantially shorter than the maximal belt circumference. For example, the band reinforcement loop lengths are from about 85 percent to about 95 percent of the maximal belt circumference.

In alternative embodiments of the invention, the first and second band reinforcements 24 and 26 differ from the above described panel reinforcements 24 and 26. For example, at least one of the first and second band reinforcements 24 and 26 may be replaced by a string allowing the intended user 13 to secure the belt 10 against the intended user. In some embodiments of the invention, the first and second band reinforcements 24 and 26 form band reinforcement closed loops secured respectively to the band lower and upper edges 20 and 22. The length of the band reinforcement closed loops are substantially shorter than a maximal circumferential length of the stretchable band closed loop formed by the stretchable band 18. In other words, when the first and second band reinforcements 24 and 26 are substantially tightly fitted against the user, the stretchable band 18 is substantially loosely fitted against the user.

In a specific example of implementation, the first and second band reinforcements 24 and 26 take the form of an elastic ribbon inserted through a passageway manufactured by folding a piece of stretchable material on itself and thereafter sewing the folded portion of the piece of stretchable material to the unfolded portion of the piece of stretchable material, the unfolded portion of the stretchable material forming the stretchable band 18.

In some embodiments of the invention, the stretchable band closed loop formed by the stretchable band 18 is circumferentially uninterrupted. In other words, the belt 10 is permanently closed and is not openable by the intended user 13, such as would occur for example if buttons, zippers or any other fasteners were provided at the two ends of a substantially rectangular piece of material.

Referring to FIG. 3, a specific example of a pocket 28 is further described. The pocket 28 includes a pocket panel 30 defining a pocket panel upper edge 32 and a pocket panel lower edge 34 laterally opposed to the pocket panel upper edge 32. In addition, first and second pocket panel side edges 36 and 38 are also defined by the pocket panel 30. The first and second pocket panel side edges 36 and 38 are circumferentially opposed to each other.

The pocket panel lower edge 32 and the first and second pocket panel side edges 36 and 38 are secured to the band inner surface 40. Therefore, the band upper edge 32 and the band inner surface 40 define a pocket aperture 44 extending substantially adjacent the panel upper edge 32, the pocket aperture 44 allowing the intended user to slip the ostomy pouch 12 into the pocket 28.

In some embodiments of the invention, the pocket panel 30 includes a substantially resilient material, such as for example, and non-limitatively, Lycra or a net.

Typically, the pocket 28 is dimensioned so that the pocket panel upper edge 34 is positionable substantially adjacent the stoma 16. In an example of implementation, the ostomy pouch 12 has a pouch lower edge 15 distanced from the stoma 16 by a stoma-to-pouch lower edge distance. The pocket panel lower edge 34 is distanced from the pocket panel upper edge 32 by a panel lower edge-to-panel upper edge distance. In this example, the panel lower edge-to-panel upper edge distance is substantially equal to the stoma-to-pouch lower edge distance. However, in alternative embodiments of the invention the pocket 28 is of any other suitable dimensions.

The belt 10 may be manufactured in any suitable manner, such as for example using stitches to form the belt from the stretchable band 18 and to secure the first and second band reinforcements 24 and 26 to the stretchable band 18. In addition, the pocket panel 30 is also securable to the stretchable band 18 using stitches. In other embodiments of the invention, any other method or device of securing materials together is used such as for example gluing the components of the belt 10 to each other.

In use, the intended user 13 simply slips the belt 10 over his legs and up to his waist 14 and positions the belt 10 so that it is comfortably held in place against the body with the pocket 28 positioned substantially in register with the ostomy pouch 12. In some embodiments of the invention, the stretchable band 18 is substantially loosely fitted relatively to the waist 14 of the intended user 13.

Then, the intended user 13 stretches the upper band reinforcement 26 and the pocket 28 to slip the ostomy pouch 12 into the pocket 28 through the aperture 44. Since the belt 10 is stretchable and does not include fasteners, it is relatively easily positioned onto the intended user's body, even by intended users 13 that have reduced mobility or who are handicapped. Furthermore, the pocket 28 maintains the ostomy pouch 12 in a substantially vertical orientation which helps in assuring proper filling of the ostomy pouch 12. Also, in embodiments of the invention wherein the pocket 28 is dimensioned so that the pocket panel upper edge 34 is positionable substantially adjacent the stoma 16, the ostomy bag 12 contacts only minimally the intended user 13, which improves air circulation between the ostomy pouch and the intended user 13.

As mentioned hereinabove, when the ostomy pouch 12 is empty, substantially no force is exerted by the stretchable band 18 onto the ostomy pouch 12, which results in substantially no pressure being exerted by the ostomy pouch 12 onto the intended user. Many factors contribute to this relative low pressure to be exerted onto the intended user 13.

Indeed, since the stretchable band 18 extends in a transversal direction over a relatively large distance, the material of the stretchable band 18 may be relatively loosely fitted against the intended user 13 and of a relatively large elasticity without causing the belt 10 to be highly mobile with respect to the intended user 13. Therefore, the use of a loosely fitted material of a relatively large elasticity results in relatively no pressure being exerted onto the intended user 13 by the ostomy pouch 12 when the ostomy pouch 12 is empty.

When the ostomy pouch 12 is filled, as shown in FIG. 4, the stretchable band 18 is stretched, which results the stretchable band 18 exerting a force onto the ostomy pouch 12 in the pressure exerting direction. This in turn creates the alert pressure 21 exerted on the intended user 13.

The reader skilled in the art will readily appreciate that in some embodiments of the invention, such as for example in embodiments wherein the stretchable band 18 has a transversal band elasticity that is substantially smaller than a circumferential band elasticity, or in embodiments of the invention wherein the stretchable band 18 is stretchable to a maximal circumferential elongation that is substantially larger than a maximal transversal elongation, the expansion of the ostomy pouch 12 results in a force, or a stress, that is concentrated in a region of the stretchable band 18 that is substantially in register with the ostomy pouch 12. Since this stress is concentrated, the stretchable band 18 may cause the exertion of the alert pressure 21 when the ostomy pouch 12 is filled while causing the exertion of a relatively low pressure or substantially no pressure when the ostomy pouch 12 is empty. The values mentioned hereinabove with respect to the band elasticity, maximal elongation of the stretchable band 18 and differences between band reinforcement loop lengths and a maximal belt circumference have been found to result in a suitable exertion of pressure onto the intended user according to whether the ostomy pouch 12 is substantially filled or substantially empty. However, the reader skilled in the art will readily appreciate that these parameters may take other values without departing from the scope of the appended claims wherein these parameters are not mentioned.

Furthermore, since the stretchable band 18 may be loosely fitted to the intended user 13 while the upper and lower band reinforcements are substantially tightly fitted to the intended user 13, a space is created between the stretchable band 18 and the intended user 13, this space allowing the insertion therein an empty ostomy pouch 12 without the exertion of a pressure onto the intended user 13.

When the intended user 13 wishes to empty the ostomy pouch 12, he simply stretches the upper band reinforcement 26 and removes the ostomy pouch 12 from the pocket 30. Since the belt 10 is relatively flexible and relatively easily stretched, this operation is relatively easily performed. Then, the intended user may empty the ostomy pouch 12 in a manner that is well known in the art. Another feature of the belt 10 is that the belt 10 protects the genitals of the intended user 13 against bodily wastes that may spill from the ostomy pouch 12 while it is emptied by the intended user 13, thereby reducing risks of infections to the genital area.

If for some reasons the ostomy pouch 12 becomes detached from the stoma 16, the pocket 28 retains the ostomy pouch 12 while exerting a relatively small pressure thereon in embodiments of the invention wherein the pocket panel 30 and the stretchable band 18 are relatively easily stretchable. This reduces risks of spillage from the ostomy pouch 12 while the intended user 13 reattaches or replaces the ostomy pouch 12.

In the embodiments of the invention shown in the drawings, the stretchable band 18 forms a stretchable band closed loop for encircling the waist 14 of the intended user. However, in alternative embodiments of the invention, a stretchable band does not form a stretchable band closed loop by itself. In these embodiments, the stretchable band is operatively coupled to another piece material that forms with the stretchable band a belt that is wearable by the intended user 13.

For example, in some embodiments of the invention, only a front portion of the belt positionable substantially adjacent to the front of the intended user 13 is stretchable, while a back portion of the belt positionable substantially in adjacent to the back portion of the intended user 13 is substantially unstretchable.

In other embodiments of the invention, a belt according to the invention includes a piece of fabric defining a fabric aperture positionable substantially in register with the ostomy pouch 12. The piece of fabric is substantially less elastic than a stretchable panel positioned substantially in register with the fabric aperture and is secured to the piece of fabric. In these embodiments, only a portion of the belt is therefore stretched when the ostomy pouch 12 is filled. In yet other alternative embodiments of the invention, the stretchable band 18 is included in a belt forming an openable belt including fasteners that allow to close and open the openable belt.

Although the present invention has been described hereinabove by way of preferred embodiments thereof, it can be modified, without departing from the spirit and nature of the subject invention as defined in the appended claims.

What is claimed is:

1. A belt for supporting an ostomy pouch, said belt being wearable by an intended user having a waist, the ostomy pouch being attachable to a stoma extending from the intended user, said belt comprising:
a stretchable band for supporting the ostomy pouch, said stretchable band being positionable substantially in register with the waist of the intended user and substantially in register with the ostomy pouch, said stretchable band defining a band lower edge and a susbtantially opposed band upper edge, said stretchable band further defining a pressure exerting direction extending substantially perpendicularly from said band towards the intended user;
a first band reinforcement operatively coupled to at least one of said band lower or upper edges for opposing, at least in part, a substantially circumferential elongation of said stretchable band;
wherein said stretchable band is configured, sized and provided with a band elasticity such that
said stretchable band exerts substantially no force onto the ostomy pouch in said pressure exerting direction when the ostomy pouch is substantially empty; and
said stretchable band exerts a band force onto the ostomy pouch in said pressure exerting direction when the ostomy pouch is substantially filled, the band force causing an alert pressure to be exerted onto the intended user by the ostomy pouch, the alert pressure being of a magnitude sufficient for alerting the intended user that the pouch is substantially filled.

2. A belt as defined in claim 1, wherein said first band reinforcement is operatively coupled to said band lower edge for opposing at least in part a substantially circumferential elongation of said stretchable band, said belt further comprising a second band reinforcement operatively coupled to said band upper edge for opposing at least in part a substantially circumferential elongation of said stretchable band.

3. A belt as defined in claim 2, wherein said stretchable band forms a stretchable band closed loop for encircling the waist of the intended user.

4. A belt as defined in claim 3, wherein said band elasticity includes a transversal band elasticity and a circumferential band elasticity, said transversal band elasticity being substantially smaller than said circumferential band elasticity.

5. A belt as defined in claim 3, wherein said stretchable band is stretchable transversally up to a maximal transversal elongation and is stretchable circumferentially up to a maximal circumferential elongation, said maximal circumferential elongation being substantially larger than said maximal transversal elongation.

6. A belt as defined in claim 5, wherein said maximal circumferential elongation is from about 1.1 to about 2 times larger than said maximal transversal elongation.

7. A belt as defined in claim 6, wherein said maximal circumferential elongation is from about 100 percent to about 200 percent and said maximal transversal elongation is from about 50 percent to about 150 percent.

8. A belt as defined in claim 7, wherein said maximal circumferential elongation is about 150 percent and said maximal transversal elongation is about 110 percent.

9. A belt as defined in claim 3, wherein said stretchable band includes Lycra™.

10. A belt as defined in claim 3, wherein said stretchable band extends transversally over a distance of at least 20 centimeters.

11. A belt as defined in claim 3, wherein said first band reinforcement is resilient and has a reinforcement elasticity, said reinforcement elasticity being substantially smaller than said band elasticity.

12. A belt as defined in claim 11, wherein:
said stretchable band closed loop defines a maximal belt circumference; and
said first band reinforcement forms a first reinforcement closed loop secured to said band upper edge, said first reinforcement closed loop having a loop length that is substantially shorter than said maximal belt circumference.

13. A belt as defined in claim 12, wherein said loop length is from about 85 percent to about 95 percent of said maximal belt circumference.

14. A belt as defined in claim 3, wherein said belt is circumferentially uninterrupted.

15. A belt as defined in claim 2, further comprising a pocket positionable between said stretchable band and the intended user, said pocket being provided for receiving therein the ostomy pouch substantially vertically.

16. A belt as defined in claim 15, wherein:
said pocket includes a pocket panel defining a pocket panel upper edge, a pocket panel lower edge substantially laterally opposed to said pocket panel upper edge, and first and second pocket panel side edges, said first pocket panel side edge being substantially circumferentially opposed to said second pocket panel side edge;
said stretchable band defines a band inner surface and an opposed band outer surface, said pocket panel lower edge and said first and second pocket panel side edges being secured to said band inner surface; and
said pocket panel upper edge and said stretchable band define an aperture allowing the intended user to insert the ostomy pouch into said pocket.

17. A belt as defined in claim 16, wherein:
the ostomy pouch has a pouch lower edge distanced from the stoma by a stoma-to-pouch lower edge distance;
said pocket panel lower edge is distanced from said pocket panel upper edge by a panel lower edge-to-panel upper edge distance; and
said panel lower edge-to-panel upper edge distance is substantially equal to said stoma-to-pouch lower edge distance.

18. A belt as defined in claim 16, wherein said pocket panel includes a substantially resilient material.

19. A belt for supporting an ostomy pouch, said belt being wearable by an intended user having a waist, the ostomy pouch being attachable to a stoma extending from the intended user, said belt comprising:
a stretchable panel for supporting the ostomy pouch, said stretchable panel being positionable substantially in register with the waist of the intended user and substantially in register with the ostomy pouch, said stretchable panel defining a panel lower edge and a susbtantially opposed panel upper edge, said stretchable panel further defining a pressure exerting direction extending substantially perpendicularly from said panel towards the intended user;
a first panel reinforcement operatively coupled to at least one of said panel lower or upper edges for opposing, at least in part, a substantially circumferential elongation of said stretchable panel;
wherein said stretchable panel is configured, sized and provided with a band elasticity such that
said stretchable panel exerts substantially no force onto the ostomy pouch in said pressure exerting direction when the ostomy pouch is substantially empty; and
said stretchable band exerts a panel force onto the ostomy pouch in said pressure exerting direction when the ostomy pouch is substantially filled, the panel force causing an alert pressure to be exerted onto the intended user by the ostomy pouch, the alert pressure being of a magnitude sufficient for alerting the intended user that the pouch is substantially filled.er, said belt comprising:
